(19) United States
(12) Patent Application Publication
(10) Pub. No.: US 2025/0288187 A1
(43) Pub. Date: Sep. 18, 2025

(54) MEDICAL OBSERVATION SYSTEM, LIGHT SOURCE DEVICE, LIGHT GUIDE CABLE, AND OBSERVATION DEVICE

(71) Applicant: Sony Olympus Medical Solutions Inc., Tokyo (JP)

(72) Inventor: Tatsuya NAKANISHI, Tokyo (JP)

(73) Assignee: Sony Olympus Medical Solutions Inc., Tokyo (JP)

(21) Appl. No.: 19/071,829

(22) Filed: Mar. 6, 2025

(30) Foreign Application Priority Data
Mar. 14, 2024 (JP) 2024-040399

(51) Int. Cl. A61B 1/00; A61B 1/04; A61B 1/06; A61B 1/07; A61B 90/00

(52) U.S. Cl. CPC A61B 1/00126; A61B 1/00117; A61B 1/043; A61B 1/0653; A61B 1/0669; A61B 1/07; A61B 90/361

(57) ABSTRACT

A technique advantageous for adaptively providing light having characteristics corresponding to each of a plurality of types of devices (including an endoscope) connectable to a light source device to a medical device connected to the light source device.

A medical observation system includes: a light guide cable that guides illumination light to a device; a light source that emits the illumination light and connection detection light toward the light guide cable; and a light detection unit that detects a wavelength of return light of the connection detection light; and a connection determination unit that determines at least one of presence or absence of connection between the light guide cable and the device and presence or absence of connection between the light guide cable and the light source on the basis of the wavelength of return light of the connection detection light.

### CROSS-REFERENCE TO RELATED APPLICATION

This application claims the benefit of Japanese Priority Patent Application JP 2024-040399 filed on Mar. 14, 2024, the entire contents of which are incorporated herein by reference.

### TECHNICAL FIELD

The present disclosure relates to a medical observation system, a light source device, a light guide cable, and an observation device.

### BACKGROUND ART

A light source device used for an endoscope such as a rigid endoscope may be provided to be connectable to other medical observation devices. For example, when a laparotomy is performed, a light source device may be connected to a medical illumination device such as a ring light. In this case, the light emitted from the light source device is emitted as illumination light from the medical illumination device, and can illuminate an entire operative field.

### CITATION LIST

### Patent Literature

[PTL 1] JP 2001-321338A

### SUMMARY

### Technical Problem

However, due to a difference in application, the characteristics of the illumination light may be changed between a case where the light source device is connected to the endoscope and a case where the light source device is connected to the medical illumination device.

However, since the light source device can be connected to both the endoscope and the medical illumination device, the illumination light to be selected may be erroneously transmitted.

The present disclosure provides a technology advantageous for adaptively providing light having characteristics corresponding to each of a plurality of types of devices (including a living body observation device such as an endoscope) connectable to a light source device to a medical device connected to the light source device.

### Solution to Problem

According to the present disclosure, there is provided a medical observation system including:
a light guide cable that guides illumination light to a device;
a light source that emits the illumination light and connection detection light toward the light guide cable; and
a light detection unit that detects a wavelength of return light of the connection detection light; and
a connection determination unit that determines at least one of presence or absence of connection between the light guide cable and the device and presence or absence of connection between the light guide cable and the light source on the basis of the wavelength of return light of the connection detection light.

A reflective member including a wavelength conversion member may be provided at a connection portion of the device with the light guide cable or the light guide cable, and the wavelength conversion member may convert a wavelength of the connection detection light and reflect the connection detection light.

A reflective member including the wavelength conversion member may be provided at a connection portion of the device with the light guide cable.

A wavelength conversion member that converts a wavelength into a wavelength different for each device may be included, and the connection determination unit may determine a type of the device connected to the light guide cable on the basis of the wavelength of return light of the connection detection light.

A first imaging device is an endoscope, and a second imaging device is an exoscope, and
the connection determination unit may determine whether there is no connection, connection to a rigid endoscope, or connection to the exoscope on the basis of the wavelength of return light of the connection detection light.

The type of the device may be determined on the basis of presence or absence of a change in the wavelength of return light of the connection detection light.

The wavelength conversion member may have a heat-resistant temperature of 115° C. or higher.

In the wavelength conversion member, phosphor may be held by a light-transmissive material having a heat-resistant temperature of 115° C. or higher.

The phosphor may be covered with a cover glass, and the phosphor may be sealed with solder.

The light detection unit may include a spectroscopic unit that reflects at least a part of return light of a first wavelength band having the same wavelength as that at the time of emission, and transmits at least a part of return light of a second wavelength band different from the first wavelength band after wavelength conversion,
a first detection unit that detects return light of the first wavelength band and return light of the second wavelength band that do not pass through the spectroscopic unit, and
a second detection unit that detects light in the second wavelength band that has passed through the spectroscopic unit, and
the connection determination unit may determine a connected device on the basis of detection results of the first detection unit and the second detection unit.

The light detection unit may include a spectroscopic unit that transmits at least a part of return light of a first wavelength band having the same wavelength as that at the time of emission, and reflects at least a part of return light of a second wavelength band different from the first wavelength band after wavelength conversion, a first detection unit that detects return light in the first wavelength band that has passed through the spectroscopic unit, and a second detection unit that detects return light in the second wavelength band that has been reflected by the spectroscopic unit, and the connection determination unit may determine a connected device on the basis of detection results of the first detection unit and the second detection unit.

The light guide cable may include a connection detection transmission path and an illumination light transmission path that is a path different from the connection detection transmission path.

The connection detection transmission path may be an optical fiber bundle forming an annular shape, the reflective member may have an annular shape, and the illumination light transmission path may be arranged at the center of an annular light guide cable.

The connection detection transmission path may be arranged in a part of a circumferential direction of the illumination light transmission path.

A reflective member including the wavelength conversion member may be provided at a connection portion of the light guide cable with the light source.

The connection determination unit may determine connection between the light guide cable and the device.

The connection determination unit may determine connection between the light guide cable and the light source.

To solve the above problem, according to the present disclosure, there is provided a medical light source device including:
an emission unit that emits illumination light and connection detection light toward a light guide cable that guides the illumination light and the connection detection light to a device;
a light detection unit that detects a wavelength of return light of the connection detection light; and
an output unit outputs information related to a wavelength of the return light to a connection determination unit that determines at least one of presence or absence of connection between the light guide cable and the device and presence or absence of connection between the light guide cable and the light source on the basis of the wavelength of return light of the connection detection light.

To solve the above problem, according to the present disclosure, there is provided a light guide cable including:
an illumination light transmission path that guides illumination light emitted from a light source to a device; and
a connection detection transmission path that guides the connection detection light emitted from the light source to the device, in which
the connection detection transmission path transmits return light of the connection detection light from the device to a return light detection unit that detects a wavelength of return light of the connection detection light in order to determine at least one of presence or absence of connection between the light guide cable and the device and presence or absence of connection between the light guide cable and the light source.

To solve the above problem, according to the present disclosure, there is provided a medical observation device including:
an illumination light incident unit on which illumination light emitted from a light source is incident through a light guide cable; and
a reflective member configured to cause return light of the connection detection light to be incident on a return light detection unit that detects a wavelength of the return light in order to determine at least one of presence or absence of connection between the light guide cable and the observation device and presence or absence of connection between the light guide cable and the light source by converting and reflecting a wavelength of the connection detection light emitted from the light source. The light guide cable may be detachable from the medical observation device, and the reflective member may be provided at a connection portion of the imaging device with the light guide cable.

A main body of the medical observation device and the light guide cable may be provided, and the reflective member may be provided at a connection portion of the light guide cable with the light source.

### BRIEF DESCRIPTION OF DRAWINGS

FIG. 1 is a diagram illustrating an example of a medical observation system.

FIG. 2 is a conceptual diagram illustrating a usage example of a medical observation system configured as an endoscope device.

FIG. 3 is a conceptual diagram illustrating a usage example of a medical observation system configured as an operative field illumination observation device.

FIG. 4 is a cross-sectional view of a light guide cable.

FIG. 5 is a block diagram illustrating a functional configuration example of a medical observation system including a rigid endoscope.

FIG. 6 is a block diagram illustrating a functional configuration example of a medical observation system including a ring light.

FIG. 7 is a block diagram illustrating a configuration example of an optical connection portion.

FIG. 8 is a diagram illustrating an example in which a reflective member is configured by only a mirror surface.

FIG. 9 is a diagram illustrating an example in which a reflective member includes a phosphor as a wavelength conversion member.

FIG. 10 is a diagram illustrating optical characteristics of a dichroic mirror.

FIG. 11 is a diagram illustrating a second configuration example of a light detection unit.

FIG. 12 is a diagram illustrating a second configuration example of the light detection unit in a case of including a phosphor.

FIG. 13 is a diagram illustrating optical characteristics of a dichroic mirror in the second configuration example of the light detection unit.

FIG. 14 is a diagram illustrating a configuration example of a reflective member according to a third embodiment.

FIG. 15 is a diagram illustrating an example of a cross section of a light guide cable according to a fourth embodiment.

FIG. 16 is a diagram schematically illustrating a connection example when a light guide cable is connected to a light source device.

FIG. 17 is a block diagram illustrating a configuration example of a reflective member according to a fifth embodiment.

### DESCRIPTION OF EMBODIMENTS

Hereinafter, embodiments of the present disclosure will be described with reference to the drawings.

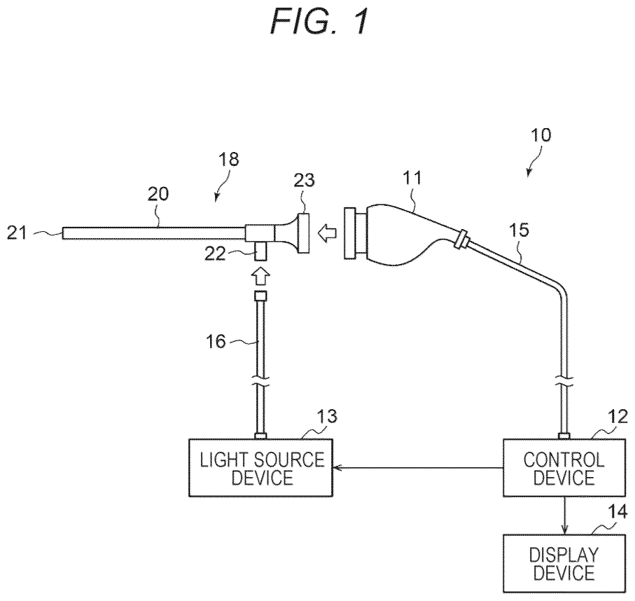

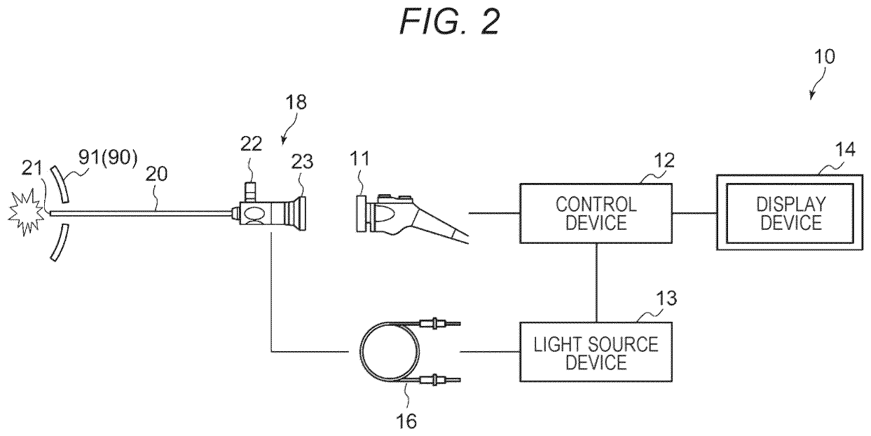

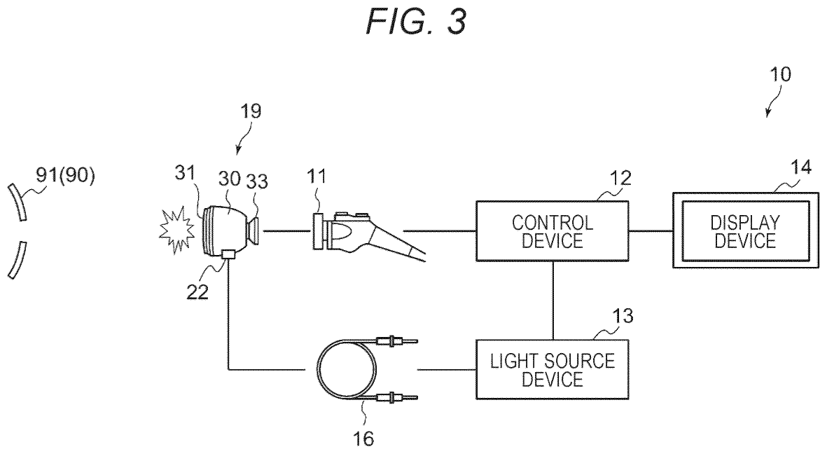

FIG. 1 is a diagram illustrating an example of a medical observation system 10, and particularly illustrates a case where the medical observation system is configured as an endoscope device including a rigid endoscope (medical observation device for endoscope for living body observation) 18. FIG. 2 is a conceptual diagram illustrating a usage example (particularly, a light emission example from the rigid endoscope 18) of the medical observation system 10 configured as an endoscope device. FIG. 3 is a conceptual diagram illustrating a usage example of the medical observation system 10 configured as an operative field illumination observation device in which a ring light (medical observation device for visual observation for living body observation) 19 is connected to a light source device 13. Note that the ring light 19 according to the present embodiment corresponds to an exoscope.

The medical observation system 10 is used to observe a target part of a subject 90 such as a patient through a captured image or to observe the target site with naked eyes. The medical observation system 10 illustrated in FIG. 1 includes an imaging device 11, a control device 12, a light source device 13, and a display device 14. The light source device 13 is provided to be connectable to the rigid endoscope 18 (see FIGS. 1 and 2) and a ring light 19 (see FIG. 3), and emits light under the control of the control device 12. The light source device 13 can emit light in an arbitrary wavelength range, and can have an arbitrary device configuration capable of emitting white light and/or narrow band light, for example. The light source device 13 is configured to also emit light for determining a device connected to a light guide cable 16. Note that the rigid endoscope 18 and the ring light 19 according to the present embodiment may be referred to as devices or imaging devices.

The white light referred to herein is light containing visible light components of various colors, and specific spectral characteristics (wavelength distribution) are not limited as long as the light can be perceived as white. On the other hand, the narrow band light includes light in a specific wavelength region of the visible light wavelength region and the invisible light wavelength region as a main light component, and has an arbitrary spectral characteristic based on a center wavelength (peak wavelength). The light source device 13 may emit, as narrowband light, excitation light (for example, infrared light) for exciting a fluorescent staining reagent used for staining a tissue (cell) to be observed and emitting fluorescence, for example.

The rigid endoscope 18 of this example is connected to the light source device 13 through the detachable light guide cable 16. That is, one end side of the light guide cable 16 is detachably attached to the light source device 13, and the other end side is detachably connected to an optical connection portion 22 of the rigid endoscope 18. Note that, according to the device, one end side of the light guide cable 16 may be fixed to the optical connection portion 22. In addition, the optical connection portion 22 according to the present embodiment corresponds to an illumination light incident unit. Similarly, the ring light 19 (see FIG. 3) of the present example is connected to the light source device 13 through the detachable light guide cable 16 (first type light guide unit). That is, one end side of the light guide cable 16 is detachably attached to the light source device 13, and the other end side is detachably connected to the optical connection portion 22 of the ring light 19. Note that, according to the device, one end side of the light guide cable 16 may be fixed to the optical connection portion 22.

The light source device 13 is configured to emit light for determining a device connected to the light guide cable 16 through the light guide cable 16. For example, return light of a part of light emitted through the light guide cable 16 is received again through the light guide cable 16. This configuration makes it possible to determine at least one of the presence or absence of connection between the light guide cable 16 and the device and the presence or absence of connection between the light guide cable 16 and the light source device 13. Furthermore, this configuration makes it possible to determine the type of a target device from which light is emitted from the light source device 13. Note that details will be described later.

The rigid endoscope 18 illustrated in FIGS. 1 and 2 includes an insertion portion 20, and the optical connection portion 22 and an imaging connection portion 23 provided on a proximal end side of the insertion portion 20. A light transmission unit (light guide) and an objective lens are provided on an end surface of an insertion distal end portion 21 of the insertion portion 20 located on the side opposite to the proximal end side. Light sent from the light source device 13 through the light guide cable 16 is emitted from the light transmission unit of the distal end side end surface of the insertion portion 20, and its reflected light (observation light/imaging light) enters the objective lens and is guided to the imaging connection portion 23 through the inside of the insertion portion 20.

The imaging connection portion 23 is detachably connected to a connection portion of the imaging device 11. The observation light transmitted through the objective lens is incident on the imaging device 11 through the imaging connection portion 23 and received by the imaging device 11. The imaging connection portion 23 can also function as an eyepiece unit. In a state where the imaging connection portion 23 is detached from the imaging device 11, a user such as an operator can directly view the observation light through the imaging connection portion 23.

On the other hand, the ring light 19 illustrated in FIG. 3 includes a main body portion 30, the optical connection portion 22 provided on the proximal end side of the main body portion 30, a light emitting unit 31 provided integrally with the main body portion 30, and an imaging connection portion 33. Light sent from the light source device 13 through the light guide cable 16 is emitted from the light emitting unit 31, and the reflected light (observation light/imaging light) is guided to the imaging connection portion 33 through an optical system (not illustrated) provided inside the main body portion 30.

The imaging connection portion 33 is detachably connected to the connection portion of the imaging device 11, and the observation light transmitted through the optical system enters the imaging device 11 through the imaging connection portion 33 and is received by the imaging device 11. The imaging connection portion 33 can also function as an eyepiece unit. In a state where the imaging connection portion 33 is detached from the imaging device 11, a user such as an operator can directly view the observation light through the imaging connection portion 33.

The imaging device 11 is provided to be connectable to the rigid endoscope 18 and the ring light 19, and receives observation light through the rigid endoscope 18 or the ring light 19 connected thereto. The imaging device 11 is connected to the control device 12 through a signal transmission cable 15 (see FIG. 1, omitted from FIGS. 2 and 3). A captured image corresponding to observation light received through the rigid endoscope 18 or the ring light 19 is transmitted from the imaging device 11 to the control device 12 through the signal transmission cable 15.

The control device 12 is connected to the imaging device 11, the light source device 13, and the display device 14, and controls the imaging device 11, the light source device 13, and the display device 14. Furthermore, the control device 12 can also control the rigid endoscope 18 or the ring light 19 connected to the imaging device 11 through the imaging device 11. For example, the control device 12 causes the display device 14 to display a captured image sent from the imaging device 11 or controls light emission of the light source device 13 as described later.

In a case where the rigid endoscope 18 is used in the medical observation system 10 described above (see FIG. 2), for example, the insertion distal end portion 21 of the rigid endoscope 18 is inserted into an abdominal cavity (body) inside the peritoneum 91 of the subject 90, and light is released from the insertion distal end portion 21 in the abdominal cavity. On the other hand, in a case where the ring light 19 is used (see FIG. 3), the ring light 19 emits light from the light emitting unit 31 outside the subject 90 (peritoneum 91).

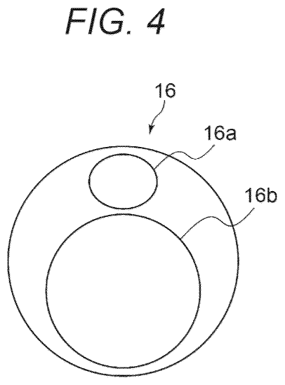

FIG. 4 is a view illustrating an example of a cross-sectional view of the light guide cable 16 according to the present embodiment. As illustrated in FIG. 4, the light guide cable 16 according to the present embodiment includes a light guide 16a for connection detection and a light guide 16b for illumination light transmission. The connection detection light guide 16a configures a connection detection transmission path. The light guide 16b for illumination light transmission configures an illumination light transmission path. As described above, the light guide cable 16 according to the present embodiment includes the connection detection optical transmission path in addition to the illumination optical transmission path in the light guide cable. Furthermore, in the light guide cable 16 according to the present embodiment, the connection detection transmission path and the illumination light transmission path are configured as different paths. As a result, the illumination light and the connection detection light are not mixed.

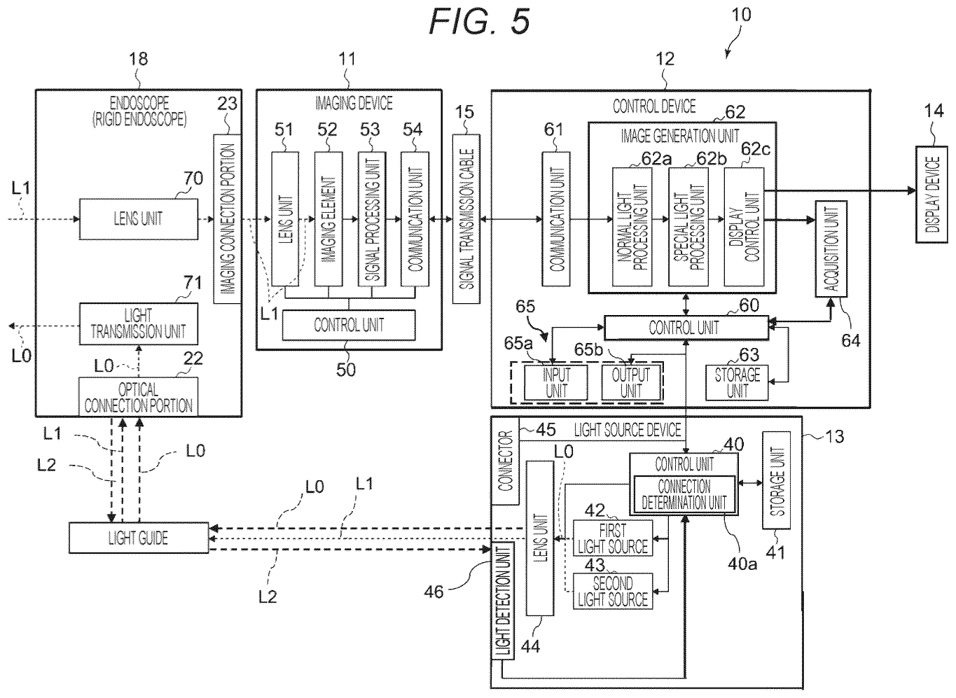

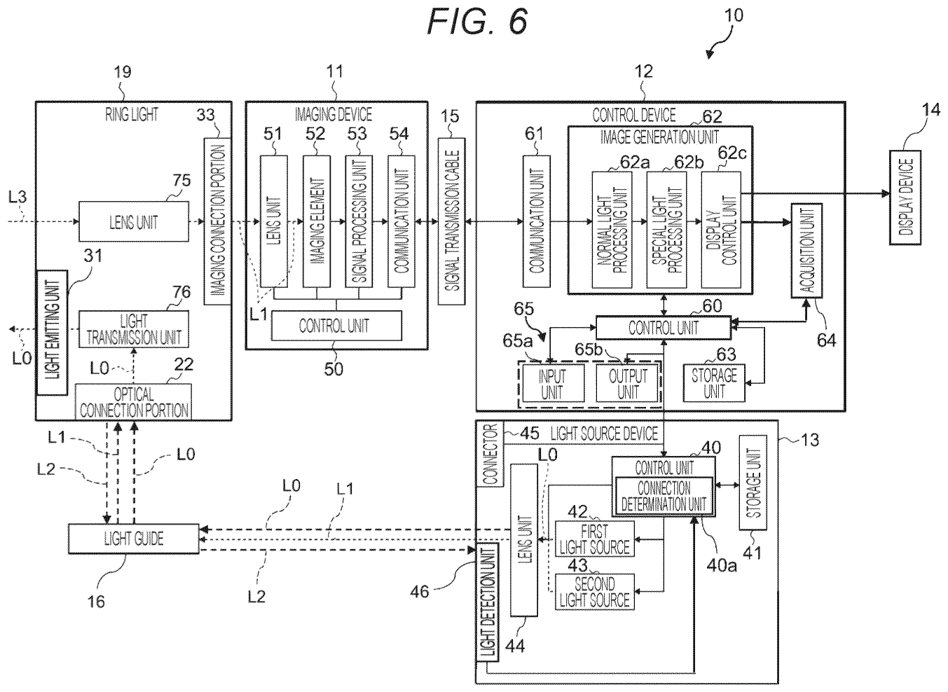

Next, a functional configuration example of the medical observation system 10 will be described. FIG. 5 is a block diagram illustrating a functional configuration example of the medical observation system (endoscope device) 10 including the rigid endoscope 18. FIG. 6 is a block diagram illustrating a functional configuration example of the medical observation system (operative field illumination observation device) 10 including the ring light 19.

In the medical observation system 10 (the endoscope device and the operative field illumination observation device) illustrated in FIGS. 5 and 6, the imaging device 11, the control device 12, and the light source device 13 have the same configuration. That is, the medical observation system 10 configures an endoscope device (see FIG. 2) by connecting the rigid endoscope 18 to the imaging device 11 and the light source device 13, and configures an operative field illumination observation device (see FIG. 3) by connecting the ring light 19 to the imaging device 11 and the light source device 13.

The light source device 13 illustrated in FIG. 5 includes a control unit 40, a storage unit 41, a first light source 42, a second light source 43, a lens unit 44, a connector 45, and a light detection unit 46. The control unit 40 of the light source device 13 controls the first light source 42, the second light source 43, and the lens unit 44 under the control of the control device 12 (particularly, the control unit 60). In this example, the lens unit 44 is divided into a lens unit for the light guide 16a (see FIG. 4) and a lens unit for the light guide 16b (see FIG. 4). Note that the lens unit 44 according to the present embodiment corresponds to an emission unit.

The first light source 42 emits white light, and the second light source 43 emits narrow band light. The connection detection light emitted from the first light source 42 and the second light source 43 passes through the lens unit for the light guide 16a (see FIG. 4) in the lens unit 44 and travels toward the connection detection light guide 16a (see FIG. 4) connected to the connector 45. The connection detection light traveling through an optical path L1 in the light guide 16a is reflected by a reflective member of the optical connection portion 22. The return light reflected by the reflective member is received by the light detection unit 46 through an optical path L2 (L1). Note that the emission of the white light in the first light source 42 and the emission of the narrow band light in the second light source 43 may be performed simultaneously, alternately, or only one of them may be performed. For example, when connection is detected, white light is emitted from first light source 42.

The light detection unit 46 outputs a reception signal corresponding to the wavelength of the return light to a connection determination unit 40a of the control unit 40. The connection determination unit 40a determines at least one of the presence or absence of connection between the light guide cable 16 and a device (rigid endoscope 18, ring light 19) and the presence or absence of connection between the light guide cable 16 and the light source 42 on the basis of the wavelength of the return light of the connection detection light. Furthermore, the connection determination unit 40a determines whether there is no connection, connection to the rigid endoscope 18, or connection to the ring light 19 on the basis of the wavelength of the return light of the connection detection light. Note that details of the light detection unit 46 will also be described later.

Similarly, the light emitted by the first light source 42 and the second light source 43 travels through the lens unit for the light guide 16b (see FIG. 4) in the lens unit 44 toward the light guide 16b (see FIG. 4) for illumination light transmission connected to the connector 45. The illumination light traveling through the optical path L0 in the light guide 16b is emitted to the body through the optical connection portion 22 and a light transmission unit 71. Note that the emission of the white light in the first light source 42 and the emission of the narrow band light in the second light source 43 may be performed simultaneously, alternately, or only one of them may be performed. In addition, a light source different from the light source used to emit the connection detection light may be used to emit the illumination light.

Furthermore, the control unit 40 accesses the storage unit 41 as necessary, reads information (which may include data and a program) from the storage unit 41, and stores new information in the storage unit 41.

The imaging device 11 illustrated in FIG. 5 includes a control unit 50, a lens unit 51, an imaging element 52, a signal processing unit 53, and a communication unit 54. Observation light (imaging light) L1 incident on the imaging device 11 is guided by the lens unit 51 and received by the imaging element 52. The imaging element 52 includes, for example, a complementary metal-oxide-semiconductor (CMOS) image sensor or a charge-coupled device (CCD) image sensor. The captured image output from the imaging element 52 that has received the observation light L1 is subjected to image processing (signal processing) in the signal processing unit 53, and then sent to the control device 12 through the communication unit 54.

The lens unit 51, the imaging element 52, the signal processing unit 53, and the communication unit 54 are driven under the control of the control unit 50. The control unit 50 of the imaging device 11 is controlled by the control device 12 (particularly, the control unit 60).

The control device 12 illustrated in FIG. 5 includes a control unit 60, a communication unit 61, an image generation unit 62, a storage unit 63, an acquisition unit 64, and a touch panel 65. Also, the touch panel 65 includes an input unit 65a and an output unit 65b. Note that the touch panel 65 according to the present embodiment corresponds to a display unit.

The captured image received from the communication unit 54 of the imaging device 11 through the signal transmission cable 15 and the communication unit 61 undergoes various processes in the image generation unit 62. For example, in a case where the captured image is a normal image based on white light, the captured image is subjected to arbitrary image processing by a normal light processing unit 62a of the image generation unit 62, then processed into a display image by a display control unit 62c, and the display image is output to the display device 14 and the acquisition unit 64. In addition, in a case where the captured image is an excitation light emission image (fluorescence image) of the fluorescent staining reagent, the captured image is subjected to arbitrary image processing by a special light processing unit 62b of the image generation unit 62, then processed into a display image by the display control unit 62c, and the display image is output to the display device 14 and the acquisition unit 64. The display device 14 displays the display image received from the image generation unit 62. Data used and/or generated by the image generation unit 62 (each of the normal light processing unit 62a to the display control unit 62c) is sent to the control unit 60 as necessary.

The control unit 60 controls the image generation unit 62, the storage unit 63, the acquisition unit 64, and the touch panel 65. The acquisition unit 64 acquires irradiation environment information regarding an irradiation environment. That is, the acquisition unit 64 can acquire a determination signal of the connection determination unit 40a and determine which one of the rigid endoscope 18 connected to the light source device 13 and the ring light 19 is emitting light.

For example, an instruction and information input by a user through the input unit 65a are sent to the control unit 60, and are appropriately used for control by the control unit 60. Furthermore, the output unit 65b is driven under the control of the control unit 60, and outputs visual information (display information) and audio information so as to present various types of information to the user. Furthermore, the control unit 60 accesses the storage unit 63 as necessary, reads information (which may include data and a program) from the storage unit 63, and stores new information in the storage unit 63.

The rigid endoscope 18 illustrated in FIG. 5 includes a lens unit 70 and the light transmission unit (light guide) 71 in addition to the optical connection portion 22 and the imaging connection portion 23 described above. The illumination light L0 (white light and/or narrow band light) sent from the light source device 13 through the light guide cable 16 passes through the optical connection portion 22, is guided by the light transmission unit 71, and is emitted from the end surface of the insertion distal end portion 21 (see FIG. 2) of the insertion portion 20. On the other hand, an observation light L3 reflected from the observation target and incident on the rigid endoscope 18 is guided by the lens unit 70, passes through the imaging connection portion 23, is then guided by the lens unit 51 of the imaging device 11, and is received by the imaging element 52.

On the other hand, the ring light 19 illustrated in FIG. 6 includes a lens unit 75 and a light transmission unit (light guide) 76 in addition to the optical connection portion 22 and the imaging connection portion 23 described above. The illumination light L0 (white light and/or narrow band light) transmitted from the light source device 13 through the light transmission unit 76 is guided by the light transmission unit 76 and emitted from the light emitting unit 31. On the other hand, the observation light L3 reflected from the observation target and incident on the ring light 19 is guided by the lens unit 75, passes through the imaging connection portion 33, is then guided by the lens unit 51 of the imaging device 11, and is received by the imaging element 52.

### (Configuration Example of Optical Connection Portion)

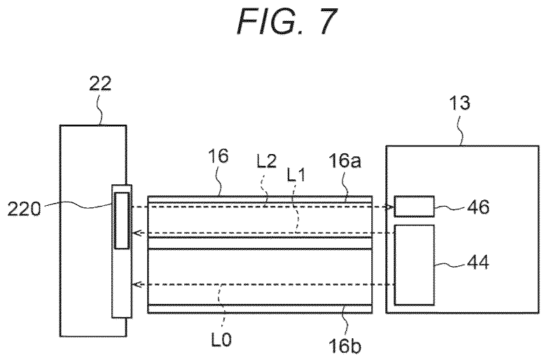

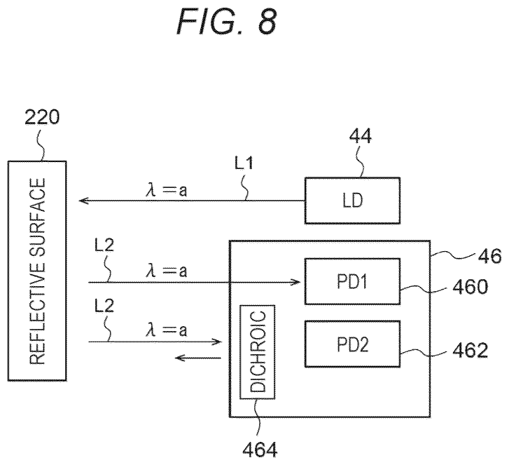

FIG. 7 is a block diagram illustrating a configuration example of the optical connection portion 22. As illustrated in FIG. 7, the optical connection portion 22 includes a reflective member 220. In this manner, the reflective member 220 is provided in the optical connection portion 22 with the detachable light guide cable 16. The reflective member 220 reflects light of different wavelengths according to a type of a device having the optical connection portion 22. That is, the type of the device connected to the optical connection portion 22 is associated with the wavelength of the reflected light reflected by the reflective member 220. For example, in the rigid endoscope 18, the reflective surface is configured by a mirror surface. Details will be described later with reference to FIG. 8. On the other hand, in the reflective member 220 of the ring light 19, the incident light is reflected by the reflective surface through, for example, a phosphor as a wavelength conversion member 220a. As described above, a plurality of phosphors can be used as the phosphor according to the type of the device to be connected. As a result, the wavelength conversion member 220a converts the incident light into reflected light having a different wavelength for each device. As described above, the reflective member 220 can change the wavelength of the reflected light to a different wavelength for each device. Note that the reflective member 220 of the rigid endoscope 18 may include the phosphor, and the reflective member 220 of the ring light 19 may not include the phosphor.

As illustrated in FIG. 7, the light guide cable 16 includes an illumination light transmission light guide 16b that guides illumination light emitted from the light source 42 (43) to the optical connection portion 22 of the imaging devices 18 and 19 through the lens unit 44, and a connection detection light guide 16a that guides connection detection light emitted from the light source 42 (43) to the optical connection portion 22 of the imaging devices 18 and 19 through the lens unit 44. With such a configuration, the connection detection light guide 16a transmits the return light L2 of the connection detection light from the imaging devices 18 and 19 to the light detection unit 46 in order to determine at least one of the presence or absence of connection between the light guide cable 16 and the imaging devices 18 and 19 and the presence or absence of connection between the light guide cable 16 and the light source 42 (43).

As described above, the connection detection light traveling through the optical path L1 of the light guide 16a for connection detection of the light guide cable 16 through the lens unit 44 is reflected by the reflective member 220 of the optical connection portion 22. The return light reflected by the reflective member 220 is received by the light detection unit 46 through the optical path L2 of the light guide 16a.

On the other hand, as described above, in the rigid endoscope 18, the illumination light traveling through the optical path L0 of the light guide 16b for transmission of illumination light through the lens unit 44 is emitted from the end surface of the insertion distal end portion 21 (see FIG. 2) of the insertion portion 20 through the lens unit 70 in addition to the optical connection portion 22 and the imaging connection portion 23 (see FIG. 5). Similarly, as described above, in the ring light 19, the illumination light traveling through the optical path L0 of the light guide 16b for transmission of illumination light through the lens unit 44 is emitted from the light emitting unit 31 through the lens unit 75 and the light transmission unit (light guide) 76 in addition to the optical connection portion 22 and the imaging connection portion 23 (see FIG. 6).

### (First Configuration Example of Light Detection Unit)

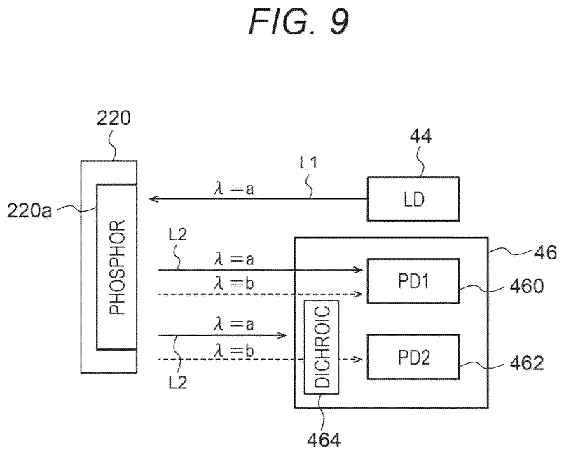

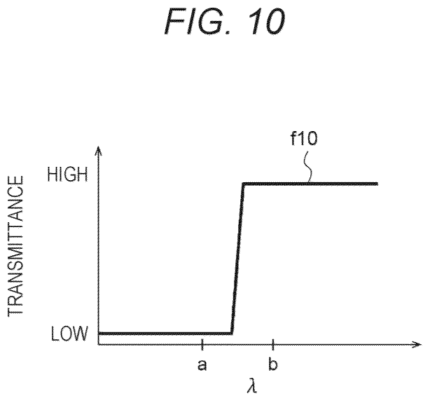

Here, a first configuration example of the light detection unit 46 will be described with reference to FIGS. 8 to 10. FIG. 8 is a diagram illustrating an example in which the reflective member 220 includes only a mirror surface. FIG. 9 is a diagram illustrating an example in which the reflective member 220 includes a phosphor as wavelength conversion member 220a. FIG. 10 is a diagram illustrating an optical characteristic f10 of a dichroic mirror. A horizontal axis indicates the wavelength λ, and a vertical axis indicates the transmittance of the dichroic mirror.

As illustrated in FIGS. 8 and 9, a wavelength conversion member 220a changes wavelength λ=a of the incident light to wavelength λ=b, and reflects the incident light as return light.

As a result, in a case where the wavelength conversion member 220a is not provided, the light having the wavelength λ=a, which is the first wavelength band having the same wavelength as that at the time of emission, becomes the return light. On the other hand, in a case where the wavelength conversion member 220a is provided, in addition to the light having the wavelength λ=a, the light having the wavelength λ=b becomes the return light.

Also, the wavelength conversion member 220a is a phosphor having a heat-resistant temperature (not deformed) of 115° C. or higher. As described above, the wavelength conversion member 220a can withstand the temperature of an autoclave (about 115° C. and −135° C.).

In addition, the light detection unit 46 detects the wavelength of the return light L2 of the connection detection light L1. The light detection unit 46 includes a first detection unit 460, a second detection unit 462, and a spectroscopic unit 464. In this configuration, the two detection units 460 and 462 are arranged side by side.

The first detection unit 460 includes a light receiving element, and outputs a first light reception signal to the connection determination unit 40a (see FIGS. 5 and 6). For example, the first detection unit 460 outputs the first light reception signal as a high-level signal when receiving reflected light of a light amount of a predetermined value or more. In a case where the reflected light of the amount of light equal to or greater than the predetermined value is not received, the first light reception signal is output as a low-level signal. For example, when receiving light of reflected light λ=a or more, the first detection unit 460 outputs the first light reception signal as the high-level signal.

The second detection unit 462 includes a light receiving element, and outputs the second light reception signal to the connection determination unit 40a (see FIGS. 5 and 6). For example, the second detection unit 462 outputs the second light reception signal as a high-level signal when receiving reflected light of a light amount of a predetermined value or more. In a case where the reflected light of the amount of light equal to or greater than the predetermined value is not received, the second light reception signal is output as a low-level signal. For example, when receiving light of reflected light λ=a or more, the second detection unit 462 outputs the second light reception signal as the high-level signal.

The spectroscopic unit 464 is, for example, a dichroic mirror. As illustrated in FIG. 10, the spectroscopic unit 464 substantially reflects return light L2 whose reflected light λ has a wavelength of a or less. On the other hand, the spectroscopic unit 464 substantially transmits the return light L2 whose reflected light λ has a wavelength of b or more. As described above, the spectroscopic unit 464 transmits at least a part of the return light of the wavelength λ=a, which is the first wavelength band having the same wavelength as that at the time of emission, and reflects at least a part of the return light of the wavelength λ=b, which is wavelength-converted and is the second wavelength band different from the first wavelength band. Thus, in a case where the reflective member 220 includes the wavelength conversion member 220a, the spectroscopic unit 464 causes return light of the connection detection light to travel toward the second detection unit 462. On the other hand, in a case where the reflective member 220 does not have the wavelength conversion member 220a, the return light of the connection detection light acts so as not to substantially advance toward the second detection unit 462.

As can be seen from these, the first detection unit 460 detects the return light of the first wavelength band (λ=a) and the return light of the second wavelength band (λ=b) that do not pass through spectroscopic unit 464. The second detection unit 462 detects the light of the second wavelength band (λ=b) transmitted through the spectroscopic unit 464.

In other words, in a case where the return light L2 travels to the first detection unit 460 and the return light L2 does not substantially travel to the second detection unit 462, the wavelength of the return light is the reflected light λ=a. On the other hand, in a case where the return light of the connection detection light travels to the first detection unit 460 and the second detection unit 462, the wavelength of the return light L2 is the reflected light λ=a, b. Furthermore, in a case where the return light of the connection detection light does not travel to the first detection unit 460 and the second detection unit 462, the light guide cable 16 and the device are not connected. Alternatively, the light guide cable 16 and the light source device 13 are not connected. As described above, the light detection unit 46 can realize the configuration for determining the wavelength of the return light L2 at a lower cost than the spectrometer.

### (Determination Example of Connection Determination Unit)

The connection determination unit 40a determines at least one of the presence or absence of connection between the light guide cable 16 and the device and the presence or absence of connection between the light guide cable 16 and the light source device 13 on the basis of the wavelength of the return light of the connection detection light. That is, in a case of the wavelength λ=a of the return light of the connection detection light or the wavelengths λ=a and b, the connection determination unit 40a determines that the light guide cable 16 and the device are connected and the light guide cable 16 and the light source device 13 are connected.

On the other hand, in a case where the wavelength of the return light of the connection detection light cannot be determined (for example, in a case where the return light of the connection detection light cannot be detected), the connection determination unit 40a determines that the light guide cable 16 and the imaging device are not connected or the light guide cable 16 and the light source device 13 are not connected.

Further, the connection determination unit 40a determines the type of the imaging device connected to the light guide cable 16 on the basis of the wavelength of the return light of the connection detection light. The connection determination unit 40a determines the type of the imaging devices 18 and 19 associated with the wavelength of the return light on the basis of the wavelength of the return light of the connection detection light. For example, in a case where the wavelength λ=a is satisfied, the connection determination unit 40a determines that a device associated with the reflective member 220 that does not have the wavelength conversion member 220a is connected. On the other hand, in a case where the wavelengths λ=a, b of the return light of the connection detection light are satisfied, the connection determination unit 40a determines that the device associated with the reflective member 220 having the wavelength conversion member 220a is connected.

That is, the connection determination unit 40a determines at least one of the presence or absence of connection between the light guide cable 16 and the imaging devices 18 and 19 and the presence or absence of connection between the light guide cable 16 and the light source 42 (43) of the light source device 13 on the basis of the combination of the first light reception signal and the second light reception signal. In addition, the connection determination unit 40a determines the types of the imaging devices 18 and 19 connected to the light guide cable 16 by a combination of the first light reception signal and the second light reception signal. More specifically, the connection determination unit 40a determines that the light guide cable 16 and the imaging devices 18 and 19 are not connected in a case where both the first light reception signal and the second light reception signal are low level signals at the time of emission of the connection detection light.

Alternatively, the connection determination unit 40a determines that the light guide cable 16 and the light source 42 (43) of the light source device 13 are not connected in a case where both the first light reception signal and the second light reception signal are low level signals at the time of emission of the connection detection light. On the other hand, in a case where at least one of the first light reception signal and the second light reception signal is a high-level signal at the time of emission of the connection detection light, the connection determination unit 40a determines that the light guide cable 16 and the imaging devices 18 and 19 are connected and the light guide cable 16 and the light source 42 (43) of the light source device 13 are connected.

Further, in a case where only the first light reception signal is the high-level signal, the connection determination unit 40a determines that the wavelength λ=a of the return light of the connection detection light is satisfied, and determines that the device associated with the reflective member 220 that does not have the wavelength conversion member 220a is connected. For example, the rigid endoscope 18 is associated with the reflective member 220 including no wavelength conversion member 220a.

On the other hand, in a case where the first light reception signal and the second light reception signal are high-level signals, the connection determination unit 40a determines that the wavelengths λ=a, b of the return light of the connection detection light are satisfied, and determines that the device associated with the reflective member 220 having the wavelength conversion member 220a is connected. For example, the ring light 19 is associated with the reflective member 220 having the wavelength conversion member 220a. In other words, the connection determination unit 40a determines the types of the imaging devices 18 and 19 on the basis of the presence or absence of a change in the wavelength of the return light L2 of the connection detection light L1. The information on the correspondence relationship between the wavelength of the return light L2 and the device is stored in advance in the storage unit 41 (see FIGS. 5 and 6). In this manner, the connection determination unit 40a determines the type of the imaging devices 18 and 19 connected to the light guide cable 16 on the basis of the detection results of the first detection unit 460 and the second detection unit 462.

In addition, the connection determination unit 40a can determine whether or not the imaging devices 18 and 19 are connected to the light source 42 (43) of the light source device 13 through the light guide cable 16. As described above, since the connection determination unit 40a determines on the basis of the wavelength of the return light L2, it is also possible to determine the types of the imaging devices 18 and 19.

As described above, the light guide cable 16 includes an illumination light transmission path that guides the illumination light emitted from the light source 42 (43) to the imaging devices 18 and 19, and a connection detection transmission path that guides the connection detection light emitted from the light source 42 (43) to the imaging devices 18 and 19. With such a configuration, the connection detection transmission path transmits the return light L2 of the connection detection light from the imaging devices 18 and 19 to the light detection unit 46 in order to determine at least one of the presence or absence of connection between the light guide cable 16 and the imaging devices 18 and 19 and the presence or absence of connection between the light guide cable 16 and the light source 42 (43).

The control unit 40 (mainly the control unit 60) executes control according to the determination of the connection determination unit 40a. For example, the control unit 40 (mainly the control unit 60) stops light emission of the light sources 42 and 43 in a case where the connection determination unit 40a determines that the light guide cable 16 and the device are not connected or the light guide cable 16 and the light source device 13 are not connected.

In a case where the connection determination unit 40a determines that the rigid endoscope 18 is connected, the control unit 40 (mainly, the control unit 60) executes light emission control for the rigid endoscope 18 on the light sources 42 and 43. Similarly, in a case where the connection determination unit 40a determines that the ring light 19 is connected, the control unit 40 (mainly the control unit 60) performs light emission control for the ring light 19 on the light sources 42 and 43.

As described above, the connection determination unit 40a determines at least one of the presence or absence of connection between the light guide cable 16 and the device and the presence or absence of connection between the light guide cable 16 and the light source device 13 on the basis of the wavelength of the return light L2 of the connection detection light reflected by the reflective member 220. As a result, it is possible to determine whether or not the imaging devices 18 and 19 are connected to the light source 42 (43) of the light source device 13 through the light guide cable 16, and it is also possible to determine the types of the imaging devices 18 and 19 since the determination is made on the basis of the wavelength of the return light L2.

Note that, in the present embodiment, two types of examples in which the type of the reflective member is the presence of the wavelength conversion member 220a and the absence of the wavelength conversion member 220a have been described in detail, but a plurality of types of wavelength conversion members that convert the connection detection light into light having different wavelengths may be provided according to the type of the device. In this case, the light detection unit 46 detects return light from a plurality of types of wavelength conversion members, and the connection determination unit 40a performs determination based on the wavelength of the return light.

Furthermore, in the present embodiment, an example in which the determination of the rigid endoscope 18 and the ring light 19 is performed as the determination of the type of the device has been described in detail, but the present embodiment is not limited thereto, and the determination of the type of the rigid endoscope 18 and the determination of the type of the ring light 19 may be performed.

### Second Embodiment

A medical observation system 10 according to a second embodiment is different from the medical observation system 10 according to the first embodiment in that optical paths of a return light L2 with respect to a first detection unit 460 and a second detection unit 462 are the same until the optical path extends to a spectroscopic unit 464. Hereinafter, differences from the medical observation system 10 according to the first embodiment will be described.

### (Second Configuration Example of Light Detection Unit)

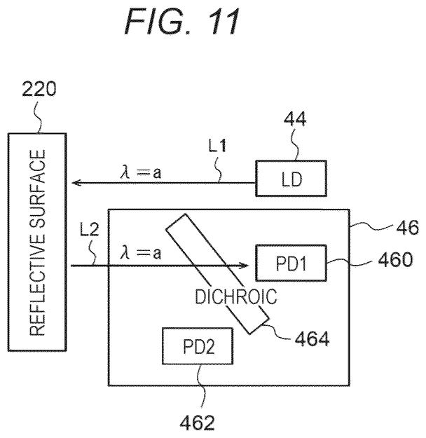

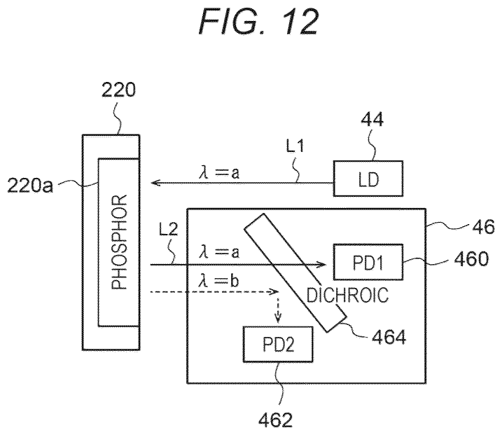

Here, a second configuration example of the light detection unit 46 will be described with reference to FIGS. 11 to 13. FIG. 11 is a diagram illustrating a second configuration example of the light detection unit 46 in a case where a reflective member 220 includes only a mirror surface. FIG. 12 is a diagram illustrating a second configuration example of the light detection unit 46 in a case where the reflective member 220 includes a phosphor as a wavelength conversion member 220a. In this configuration, the two detection units 460 and 462 are oriented 90 degrees differently from each other.

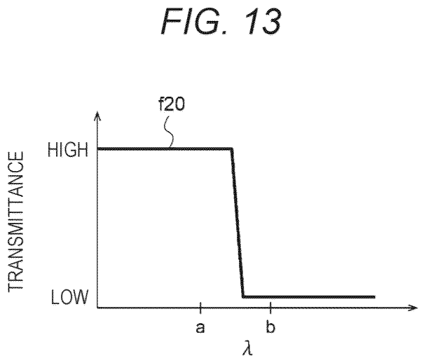

FIG. 13 is a diagram illustrating optical characteristics f20 of the dichroic mirror in the second configuration example of the light detection unit 46. A horizontal axis indicates the wavelength λ, and a vertical axis indicates the transmittance of the dichroic mirror. As illustrated in FIGS. 11 and 12, wavelength conversion member 220a changes wavelength λ=a of the incident light to wavelength λ=b, and reflects the incident light as return light.

The spectroscopic unit 464 is, for example, a dichroic mirror. As illustrated in FIG. 10, the spectroscopic unit 464 substantially transmits the return light in a case where the reflected light λ has a wavelength of a or less. On the other hand, in a case where the reflected light λ has a wavelength of b or more, the spectroscopic unit 464 substantially reflects the return light. As described above, the spectroscopic unit 464 reflects at least a part of the return light of the first wavelength band (λ=a) having the same wavelength as that at the time of emission, and reflects at least a part of the return light L2 of the second wavelength band (λ=b) that is wavelength-converted and different from the first wavelength band.

As illustrated in FIG. 11, in a case where the reflective member 220 does not include the wavelength conversion member 220a, wavelength λ=a of the return light of the connection detection light is satisfied. The second detection unit 462 is disposed on the reflective surface side of the spectroscopic unit 464, and the first detection unit 460 is disposed on the transmission side of the spectroscopic unit 464. As can be seen from the above, in a case where the wavelength λ=a of the return light is satisfied, since the return light passes through the spectroscopic unit 464, the first detection unit 460 outputs the high level signal as a first light reception signal. On the other hand, the second detection unit 462 outputs a low-level signal as a second light reception signal. In addition, the optical path of the return light to the spectroscopic unit 464 can be shared by the first detection unit 460 and the second detection unit 462. Therefore, a cross-sectional area of the optical path of the return light to the spectroscopic unit 464 can be further reduced.

As illustrated in FIG. 12, in a case where the reflective member 220 includes the wavelength conversion member 220a, wavelengths λ=a, b of the return light of the connection detection light are satisfied. The second detection unit 462 is disposed on the reflective surface side of the spectroscopic unit 464, and the first detection unit 460 is disposed on the transmission side of the spectroscopic unit 464. As can be seen from the above, in a case where the wavelengths λ=a, b of the return light are satisfied, the return light having the wavelength λ=a is transmitted through the spectroscopic unit 464, so that the first detection unit 460 outputs the high-level signal as the first light reception signal. On the other hand, since the return light having the wavelength λ=b is reflected by the spectroscopic unit 464, the second detection unit 462 outputs the high level signal as the second light reception signal. In addition, the optical path of the return light to the spectroscopic unit 464 can be shared by the first detection unit 460 and the second detection unit 462. Therefore, a cross-sectional area of the optical path of the return light to the spectroscopic unit 464 can be further reduced. Note that the first configuration and the second configuration of the light detection unit can be selected according to the shape of a space in which the light detection unit 46 can be arranged.

As in the similar manner described above, the connection determination unit 40a determines at least one of the presence or absence of connection between the light guide cable 16 and the device and the presence or absence of connection between the light guide cable 16 and the light source device 13 on the basis of the wavelength of the return light of the connection detection light. That is, in a case of the wavelength λ=a of the return light of the connection detection light or the wavelengths λ=a and b, the connection determination unit 40a determines that the light guide cable 16 and the device are connected and the light guide cable 16 and the light source device 13 are connected.

On the other hand, in a case where the wavelength of the return light of the connection detection light cannot be determined (for example, in a case where the return light of the connection detection light cannot be detected), the connection determination unit 40a determines that the light guide cable 16 and the imaging device are not connected or the light guide cable 16 and the light source device 13 are not connected.

In addition, as described above, the connection determination unit 40a determines the type of the imaging devices 18 and 19 connected to the light guide cable 16 on the basis of the wavelength of the return light of the connection detection light. For example, in a case where the wavelength λ=a is satisfied, the connection determination unit 40a determines that a device associated with the reflective member 220 that does not have the wavelength conversion member 220a is connected. On the other hand, in a case where the wavelengths λ=a, b of the return light of the connection detection light are satisfied, the connection determination unit 40a determines that the device associated with the reflective member 220 having the wavelength conversion member 220a is connected.

As described above, according to the present embodiment, the spectroscopic unit 464 having characteristics of transmitting the wavelength λ=a and reflecting the wavelength λ=b is arranged obliquely with respect to the optical path L2 of the return light, the second detection unit 462 is arranged on the reflective surface side of the spectroscopic unit 464, and the first detection unit 460 is arranged on the transmission side of the spectroscopic unit 464. As a result, the combination of the output signals of the first detection unit 460 and the second detection unit 462 can be made different between the case of the wavelengths λ=a, b of the return light and the case of the wavelength λ=a of the return light, and the wavelength of the return light can be determined. Therefore, determination similar to that of the connection determination unit 40a according to the first embodiment can be made, and the optical path of the return light to the spectroscopic unit 464 can be made common between the first detection unit 460 and the second detection unit 462, so that the cross-sectional area of the optical path of the return light to the spectroscopic unit 464 can be further reduced.

### Third Embodiment

A medical observation system 10 according to a third embodiment is different from the medical observation system 10 according to the first embodiment in that a wavelength conversion member 220a is held on a reflective member 220 by a light-transmissive material 222. Hereinafter, differences from the medical observation system 10 according to the first embodiment will be described.

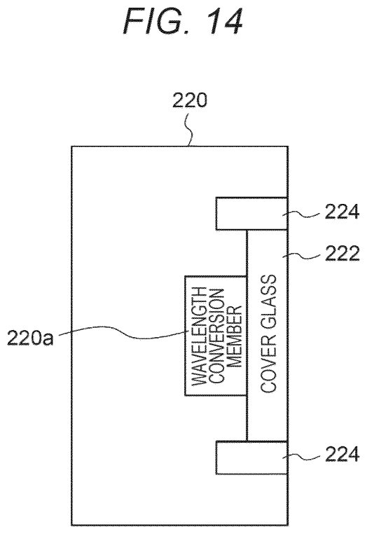

FIG. 14 is a diagram illustrating a configuration example of the reflective member 220 according to the third embodiment. The light-transmissive material 222 is, for example, cover glass, and is a light-transmissive material having a heat-resistant temperature (not deformed) of 115° C. or higher. The light-transmissive material 222 is sealed by a solder sealing portion 224, and holds the wavelength conversion member 220a on the reflective member 220.

As a result, in the configuration in which the wavelength conversion member 220a that is a phosphor is disposed on the reflective member 220 that is a resin, the configuration can withstand the temperature of an autoclave (about 115° C. and −135° C.).

### Fourth Embodiment

A medical observation system 10 according to a fourth embodiment is different from the medical observation system 10 according to the first embodiment in that a cross section of a light guide cable 16 is rotationally symmetric. Hereinafter, differences from the medical observation system 10 according to the first embodiment will be described.

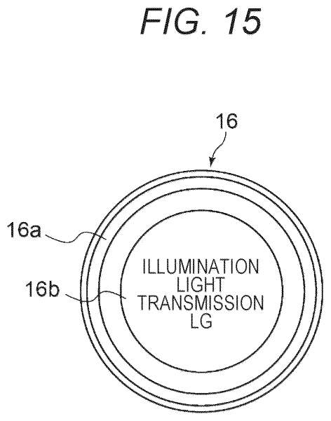

FIG. 15 is a diagram illustrating an example of a cross section of the light guide cable 16 according to the fourth embodiment. As illustrated in FIG. 15, a cross section of a connection detection light guide 16a according to the present embodiment is rotationally symmetric and has a structure without anisotropy. As a result, when the light guide cable 16 is connected to a light source device 13, connection at an arbitrary rotation angle is possible.

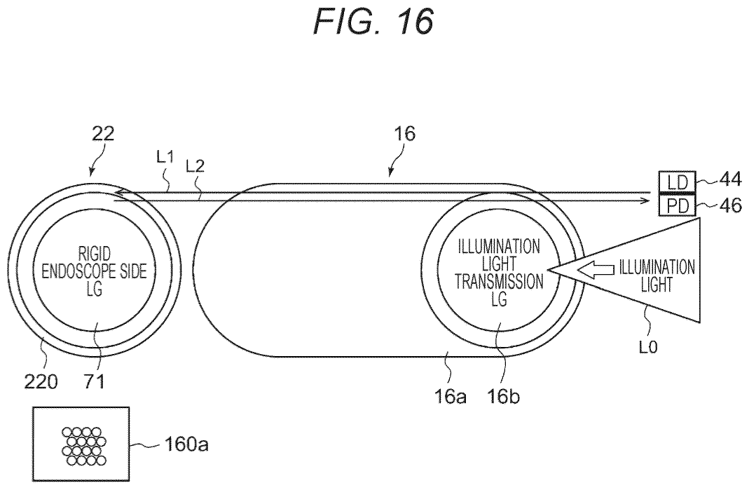

FIG. 16 is a diagram schematically illustrating a connection example when the light guide cable 16 is connected to the light source device 13 according to the fourth embodiment. As illustrated in FIG. 16, a connection detection transmission path in the light guide 16a is arranged in an annular shape so as to surround the circumference of an illumination light transmission path in the light guide 16b.

The light guide 16a and the light guide 16b are configured as, for example, a bundle of optical fibers 160a. As described above, the connection detection transmission path is the optical fiber bundle 160a forming an annular shape, the reflective member 220 also has an annular shape, and the illumination light transmission path is arranged at the center of the light guide cable 16 having an annular shape. Therefore, for example, even in a case where the light guide 16a is twisted, the return light returns to the position of the light detection unit 46. As a result, the positions of a lens unit 44 and a light detection unit 46 can be stably detected at one place. Therefore, when the light guide cable 16 is connected to an optical connection portion 22, the light guide cable 16 can be connected at an arbitrary rotation angle. In addition, the light guide 16a for connection detection has an annular shape, but the lens unit 44 and/or the light detection unit 46 are provided at one location, so that the cost can be suppressed. Note that in the light guide cable 16 of the first embodiment illustrated in FIG. 4, the connection detection transmission path in the light guide 16a is arranged in a part of the circumferential direction of the illumination light transmission path in the light guide 16b. In the light guide cable 16 of the first embodiment, anisotropy occurs in the arrangement positions of the lens unit 44 and the light detection unit 46, but the cable configuration of the light guide cable 16 can be simplified.

### Fifth Embodiment

A medical observation system 10 according to a fifth embodiment is different from the medical observation system 10 according to the first embodiment in that a reflective member 220 is formed on a connection side of a light guide cable 16 with a light source device 13. Hereinafter, differences from the medical observation system 10 according to the first embodiment will be described.

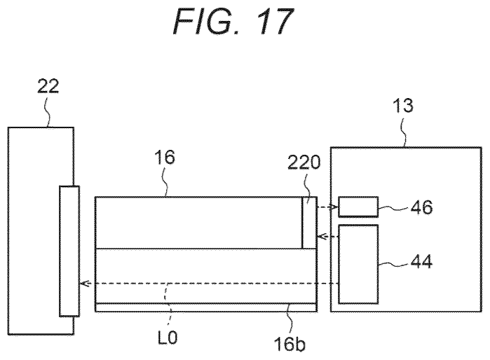

FIG. 17 is a block diagram illustrating a configuration example of the reflective member 220 according to the fifth embodiment. As illustrated in FIG. 17, the reflective member 220 is configured on a connection side of the light guide cable 16 with the light source device 13. As described above, in a case where the light guide cable 16 and the light source device 1 cannot be attached and detached, the reflective member 220 can be provided at a connection portion between the light guide cable 16 and the light source device 13. The reflective member 220 can further configure a wavelength conversion member 220a (see FIG. 9).

As described above, in a case where the light guide cable 16 and the imaging devices 18 and 19 are not detachable and attachable, the configuration of the light guide 16a becomes unnecessary. Therefore, the light guide cable 16 can be configured more easily.

The present disclosure can also have the following configurations.

(1)

A medical observation system including:
a light guide cable that guides illumination light to a device;
a light source that emits the illumination light and connection detection light toward the light guide cable;
a light detection unit that detects a wavelength of return light of the connection detection light; and
a connection determination unit that determines at least one of presence or absence of connection between the light guide cable and the device and presence or absence of connection between the light guide cable and the light source on the basis of the wavelength of return light of the connection detection light.
   (2)

The medical observation system according to (1), in which a reflective member including a wavelength conversion member is provided at a connection portion of the device with the light guide cable or the light guide cable, and the wavelength conversion member converts a wavelength of the connection detection light and reflects the connection detection light.

(3)

The medical observation system according to (2), in which the reflective member including the wavelength conversion member is provided at the connection portion of the device with the light guide cable.

(4)

The medical observation system according to (1), further including a wavelength conversion member that converts a wavelength into a different wavelength for each device, in which the connection determination unit determines a type of the device connected to the light guide cable on the basis of the wavelength of return light of the connection detection light.

(5)

The medical observation system according to (1), in which
a first imaging device is an endoscope, and a second imaging device is an exoscope, and
the connection determination unit determines whether there is no connection, connection to a rigid endoscope, or connection to the exoscope on the basis of the wavelength of return light of the connection detection light.
   (6)

The medical observation system according to (1), in which a type of the device is determined on the basis of presence or absence of a change in the wavelength of return light of the connection detection light.

(7)

The medical observation system according to (2), in which the wavelength conversion member has a heat-resistant temperature of 115° C. or higher.

(8)

The medical observation system according to (7), in which in the wavelength conversion member, a phosphor is held by a light-transmissive material having a heat-resistant temperature of 115° C. or higher.

(9)

The medical observation system according to (8), in which the phosphor is covered with a cover glass, and the phosphor is sealed with solder.

(10)

The medical observation system according to (1), in which the light detection unit includes a spectroscopic unit that reflects at least a part of return light of a first wavelength band having the same wavelength as that at the time of emission, and transmits at least a part of return light of a second wavelength band different from the first wavelength band after wavelength conversion,
a first detection unit that detects return light of the first wavelength band and return light of the second wavelength band that do not pass through the spectroscopic unit, and
a second detection unit that detects light in the second wavelength band that has passed through the spectroscopic unit, and
the connection determination unit determines a connected device on the basis of detection results of the first detection unit and the second detection unit.
   (11)

The medical observation system according to (1), in which the light detection unit includes a spectroscopic unit that transmits at least a part of return light of a first wavelength band having the same wavelength as that at the time of emission, and reflects at least a part of return light of a second wavelength band different from the first wavelength band after wavelength conversion, a first detection unit that detects return light in the first wavelength band that has passed through the spectroscopic unit, and a second detection unit that detects return light in the second wavelength band that has been reflected by the spectroscopic unit, and
the connection determination unit determines a connected device on the basis of detection results of the first detection unit and the second detection unit.
   (12)

The medical observation system according to (1), in which the light guide cable includes a connection detection transmission path and an illumination light transmission path that is a path different from the connection detection transmission path.

(13)

The medical observation system according to (12), in which the connection detection transmission path is an optical fiber bundle forming an annular shape, the reflective member also has an annular shape, and the illumination light transmission path is arranged at the center of an annular light guide cable.

(14)

The medical observation system according to (1), in which the connection detection transmission path is arranged in a part of a circumferential direction of the illumination light transmission path.

(15)

The medical observation system according to (1), in which a reflective member including the wavelength conversion member is provided at a connection portion of the light guide cable with the light source.

(16)

The medical observation system according to (1), in which the connection determination unit determines connection between the light guide cable and the device.

(17)

The medical observation system according to (1), in which the connection determination unit determines connection between the light guide cable and the light source.

(18)

A medical light source device including:
an emission unit that emits illumination light and connection detection light toward a light guide cable that guides the illumination light and the connection detection light to a device;
a light detection unit that detects a wavelength of return light of the connection detection light; and
an output unit that outputs information related to a wavelength of the return light to a connection determination unit that determines at least one of presence or absence of connection between the light guide cable and the device and presence or absence of connection between the light guide cable and the light source on the basis of the wavelength of return light of the connection detection light.
   (19)

A light guide cable including:
an illumination light transmission path that guides illumination light emitted from a light source to a device; and
a connection detection transmission path that guides connection detection light emitted from the light source to the device, in which
the connection detection transmission path transmits return light of the connection detection light from the device to a return light detection unit that detects a wavelength of return light of the connection detection light in order to determine at least one of presence or absence of connection between the light guide cable and the device and presence or absence of connection between the light guide cable and the light source.
   (20)

A medical observation device including:
an illumination light incident unit on which illumination light emitted from a light source is incident through a light guide cable; and
a reflective member configured to cause return light of connection detection light to be incident on a return light detection unit that detects a wavelength of the return light in order to determine at least one of presence or absence of connection between the light guide cable and the observation device and presence or absence of connection between the light guide cable and the light source by converting and reflecting a wavelength of the connection detection light emitted from the light source.
   (21)

The medical observation device according to (20), in which the light guide cable is detachable from the medical observation device, and the reflective member is provided at a connection portion of the imaging device with the light guide cable.

(22)

The medical observation device according to (20), further including:
a main body of the medical observation device; and
the light guide cable, in which
the reflective member is provided at a connection portion of the light guide cable with the light source.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

10 Medical observation system
11 Imaging device
13 Light source device
16 Light guide cable
16a Light guide for connection detection
16b Light guide for illumination light transmission
18 Rigid endoscope (medical observation device)
19 Ring light (medical observation device)
22 Optical connection portion
40a Connection determination unit
42 First light source
43 Second light source
44 Lens unit
160a Optical fiber
220 Reflective member
220a Wavelength conversion member
460 First detection unit
462 Second detection unit
464 Spectroscopic unit

1. A medical observation system comprising:
a light guide cable that guides illumination light to a device;
a light source that emits the illumination light and connection detection light toward the light guide cable;
a light detection unit that detects a wavelength of return light of the connection detection light; and
a connection determination unit that determines at least one of presence or absence of connection between the light guide cable and the device and presence or absence of connection between the light guide cable and the light source on a basis of the wavelength of return light of the connection detection light.

2. The medical observation system according to claim 1, wherein a reflective member including a wavelength conversion member is provided at a connection portion of the device with the light guide cable or the light guide cable, and the wavelength conversion member converts a wavelength of the connection detection light and reflects the connection detection light.

3. The medical observation system according to claim 2, wherein the reflective member including the wavelength conversion member is provided at the connection portion of the device with the light guide cable.

4. The medical observation system according to claim 1, further comprising a wavelength conversion member that converts a wavelength into a different wavelength for each device, wherein
the connection determination unit determines a type of the device connected to the light guide cable on a basis of the wavelength of return light of the connection detection light.

5. The medical observation system according to claim 1, wherein
a first imaging device is an endoscope, and a second imaging device is an exoscope, and
the connection determination unit determines whether there is no connection, connection to a rigid endoscope, or connection to the exoscope on a basis of the wavelength of return light of the connection detection light.

6. The medical observation system according to claim 1, wherein a type of the device is determined on a basis of presence or absence of a change in the wavelength of return light of the connection detection light.

7. The medical observation system according to claim 2, wherein the wavelength conversion member has a heat-resistant temperature of 115° C. or higher.

8. The medical observation system according to claim 7, wherein in the wavelength conversion member, a phosphor is held by a light-transmissive material having a heat-resistant temperature of 115° C. or higher.

9. The medical observation system according to claim 8, wherein the phosphor is covered with a cover glass, and the phosphor is sealed with solder.

10. The medical observation system according to claim 1, wherein
the light detection unit includes:
a spectroscopic unit that reflects at least a part of return light of a first wavelength band having the same wavelength as that at the time of emission, and transmits at least a part of return light of a second wavelength band different from the first wavelength band after wavelength conversion;
a first detection unit that detects return light of the first wavelength band and return light of the second wavelength band that do not pass through the spectroscopic unit; and
a second detection unit that detects light in the second wavelength band that has passed through the spectroscopic unit, and
the connection determination unit determines a connected device on a basis of detection results of the first detection unit and the second detection unit.

11. The medical observation system according to claim 1, wherein
the light detection unit includes:
a spectroscopic unit that transmits at least a part of return light of a first wavelength band having the same wavelength as that at the time of emission, and reflects at least a part of return light of a second wavelength band different from the first wavelength band after wavelength conversion;
a first detection unit that detects return light in the first wavelength band that has passed through the spectroscopic unit; and
a second detection unit that detects return light in the second wavelength band that has been reflected by the spectroscopic unit, and
the connection determination unit determines a connected device on a basis of detection results of the first detection unit and the second detection unit.

12. The medical observation system according to claim 1, wherein the light guide cable includes a connection detection transmission path and an illumination light transmission path that is a path different from the connection detection transmission path.

13. The medical observation system according to claim 12, wherein the connection detection transmission path is an optical fiber bundle forming an annular shape, the reflective member has an annular shape, and the illumination light transmission path is arranged at a center of an annular light guide cable.

14. The medical observation system according to claim 1, wherein the connection detection transmission path is arranged in a part of a circumferential direction of the illumination light transmission path.

15. The medical observation system according to claim 1, wherein a reflective member including the wavelength conversion member is provided at a connection portion of the light guide cable with the light source.

16. The medical observation system according to claim 1, wherein the connection determination unit determines connection between the light guide cable and the device.

17. The medical observation system according to claim 1, wherein the connection determination unit determines connection between the light guide cable and the light source.

18. A medical light source device comprising:
an emission unit that emits illumination light and connection detection light toward a light guide cable that guides the illumination light and the connection detection light to a device;
a light detection unit that detects a wavelength of return light of the connection detection light; and
an output unit that outputs information related to a wavelength of the return light to a connection determination unit that determines at least one of presence or absence of connection between the light guide cable and the device and presence or absence of connection between the light guide cable and the light source on a basis of the wavelength of return light of the connection detection light.

19. A light guide cable comprising:
an illumination light transmission path that guides illumination light emitted from a light source to a device; and
a connection detection transmission path that guides connection detection light emitted from the light source to the device, wherein
the connection detection transmission path transmits return light of the connection detection light from the device to a return light detection unit that detects a wavelength of return light of the connection detection light in order to determine at least one of presence or absence of connection between the light guide cable and the device and presence or absence of connection between the light guide cable and the light source.

20. A medical observation device comprising:
an illumination light incident unit on which illumination light emitted from a light source is incident through a light guide cable; and
a reflective member configured to cause return light of connection detection light to be incident on a return light detection unit that detects a wavelength of the return light in order to determine at least one of presence or absence of connection between the light guide cable and the observation device and presence or absence of connection between the light guide cable and the light source by converting and reflecting a wavelength of the connection detection light emitted from the light source.

21. The medical observation device according to claim 20, wherein the light guide cable is detachable from the medical observation device, and the reflective member is provided in the illumination light incident unit.

22. The medical observation device according to claim 20, further comprising:
a main body of the medical observation device; and
the light guide cable, wherein
the reflective member is provided at a connection portion of the light guide cable with the light source.
